(19) United States
(12) Patent Application Publication
(10) Pub. No.: US 2008/0020077 A1
(43) Pub. Date: Jan. 24, 2008

(54) Association Of Vegetal Extracts Based On Gooseberries, Black Orchids And Black Tulips And Topical Composition Comprising The Association Of Said Vegetal Extracts

(76) Inventor: Jacques Leclere, Saint-Gondon (FR)

Correspondence Address: BUCHANAN, INGERSOLL & ROONEY PC, POST OFFICE BOX 1404, ALEXANDRIA, VA, 22313-1404, US

(21) Appl. No.: 11/628,233

(22) PCT Filed: Jun. 1, 2005
(86) PCT No.: PCT/FR05/01346

§ 371 Date: Dec. 1, 2006

(30) Foreign Application Priority Data
Jun. 3, 2004 (FR) 0406003

(51) Int. Cl. A61K 36/185; A61K 36/00; A61K 36/898; A61P 17/00

(52) U.S. Cl. 424/776; 424/725

(57) ABSTRACT

The invention relates to a combination of plant extracts which can be used in a topical composition. The combination of plant extracts is based on gooseberries, black orchids and black tulips, the content thereof ranging from 5% to 90% in relation to the total weight of the combination. Application: treatment or prevention of damage to the skin associated with ageing.

The present invention relates to a combination based on gooseberry, black orchid and black tulip plant extracts, and also to a topical composition containing same, and more particularly to a composition containing such a combination, that can be used for the treatment and prevention of damage associated with skin ageing, and also to the associated cosmetic processes.

The skin comprises several integrated layers, ranging from the superficial layer, the epidermis, to the deeper layers, the dermis and the hypodermis, and each one possesses specific properties that allow the whole to react and adapt to the conditions of its environment.

The epidermis is mainly composed of three types of cells, which are keratinocytes (90% of epidermal cells), melanocytes (2 to 3% of epidermal cells) and Langerhans cells. Its thickness varies according to the various parts of the body. The epidermis, which constitutes the outer layer, plays a fundamental role in ensuring protection and maintenance of good trophicity. This is why many compositions have been developed in order to protect it and to improve its functions, and in particular to reinforce its elasticity and its firmness.

The dermis is thick, solid, and rich in nerves, in blood vessels and in sweat glands. It protects and repairs damaged tissues. This layer is composed mainly of collagen, elastin and proteoglycans. These three types of molecules are synthesized by the dermal fibroblasts. The collagen fibers, which represent 70% of the dry weight of the dermis, form a support network responsible for the mechanical characteristics of the skin, such as the mechanical strength and the texture. The elastin is responsible for the elasticity and the proteoglycans play a major role in the structure and moisturization of the skin. Other cells, such as macrophages and leucocytes, are also present in the dermal layer.

The hypodermis, which is attached at the bottom of the dermis, is the deepest layer of the skin. It contains the adipocytes which produce lipids so that the subcutaneous tissue produces a fatty layer that protects the muscles, the bones and the internal organs against impacts, and acts as an insulator and a source of energy during fasting periods.

One of the first signs of skin ageing is a loss of elasticity and the formation of wrinkles and fine lines, which are the direct result of the deterioration of the supporting dermal layer. In fact, the skin's ability to replace damaged collagen decreases and more spaces and irregularities develop in the collagen network. The appearance of pigmentary spots, thinness of the skin and sagging of the skin are also changes observed during subsequent ageing of the skin.

Numerous factors contribute to accelerating collagen degradation, and in particular exposure to the sun, free radicals, certain age-related hormonal changes, and tobacco.

Skin ageing is often described as appearing in two ways. The first is chronological or intrinsic ageing, while the second is extrinsic ageing, i.e. ageing caused by the environment; this is more particularly the case of photo ageing, which is this damage caused to the skin due to the direct or indirect effects of ultraviolet light. The present invention relates not only to intrinsic ageing, but also to extrinsic ageing.

On the scale of the skin, ageing causes in particular a decrease in protein (collagen, elastin) syntheses, a decrease in the synthesis of proteoglycans among others, and also an increase in MMP3-type metalloproteinases.

Moreover, oxidation phenomena (free radical content) lead to inflammatory processes that increase skin sensitivity.

To combat skin ageing, certain treatments have been proposed, based on creams containing alpha-hydroxy acids or retinoids, applied regularly so as to reduce in the long term the number of fine lines. Collagen implants for concealing expression lines around the eyes or the mouth, dermabrasion and chemical peels for eliminating the top layer of damaged skin, cosmetic surgery, such as blepharoplasty (eyelid surgery) or a lift for rejuvenating sagging skin, or alternatively a restructuring using a carbon dioxide laser for eliminating wrinkles and improving scars, have also been proposed.

Patent application WO 02069992 cites a large number of plant extracts obtained under specific conditions where the plant is subjected beforehand to a stress, and capable of providing an inhibitory effect on certain extracellular proteases and, consequently, of limiting the harmful effects attributed to these proteases. The use of certain plant extracts in cosmetic compositions is also described in patent application JP 02279618 relating to water-soluble extracts of orchid with a moisturizing action, patent application JP 2003040758 relating to extracts of saxifrage and patent application JP 10007582 relating to extracts of tulip.

However, there still exists an increased need to find alternative topical compositions for effectively combating skin ageing, and in particular to find topical compositions based on appropriate plant extracts, particularly originating from plants known for their abilities to resist and to live in a depleted medium or for their high nucleotide content.

According to the present invention, it has now been found, surprisingly, that it is possible to use a combination of plant extracts based on gooseberry, black orchid and black tulip, for the preparation of a topical application for use in the treatment or prevention of damage to the skin associated with ageing. In fact, it has been observed that said plant extract increases the perimembrane, transmembrane and matrix proteoglycan content and also increases the collagen content of human dermal fibroblasts. They can thus be used as anti-ageing cosmetic composition. Thus, the composition according to the invention can be applied topically to an area of the skin in order to counterbalance the loss of cell renewal and the loss of dermal functions.

A subject of the present invention is therefore a combination of plant extracts based on gooseberry, black orchid and black tulip.

A subject of the present invention is also a topical composition for the treatment or prevention of damage to the skin associated with ageing, comprising a combination according to the first subject of the invention indicated above.

An object of the invention is also to propose the use of a combination as described above, for the preparation of a topical composition for combating damage to the skin associated with ageing.

Finally, a subject of the present invention is also a cosmetic process for combating damage to the skin associated with ageing, which comprises a step consisting in applying, to affected areas of the skin, a composition containing an effective amount of the topical composition according to the second subject of the present invention.

The cosmetic process for improving the external appearance, comprising a step consisting in applying this topical composition to affected areas of the skin, also constitutes another subject of the present invention.

These topical compositions may advantageously be used in dermatology or cosmetology for the treatment or prevention of damage to the skin associated with ageing, more particularly comprising the loss of firmness and of tonicity of the skin, and the appearance of wrinkles and fine lines.

In the subsequent text, in the interests of simplification, the topical composition is of useful dermatological or cosmetic purposes, unless otherwise indicated.

Further details are given hereinafter regarding the plants from which are derived the plant extracts on which the combination according to the present invention is based.

The gooseberry is in particular known for its high polyphenol content (Ribes uva-crispa). The gooseberry originates from Asia and has become subspontaneous in Northern and Eastern Europe. It is found in the planes or at the bottom of mountains, colonizing poor environments or else terrains that are specifically acidic. For example, it is found in les Vosges up to an altitude of 1000 m and also on the embankments of the Loire in areas liable to flooding. It is the fruit that is used.

The black orchid (Cycnoches cooperi), like all orchids, lives in a poor environment and pine bark or lichen debris may be sufficient for its development. The only essential elements that are required are light, moisture and warmth. It is capable of storing nutritive substances and moisture. The black orchid is particularly rich in flavonoids, which are particularly advantageous as free-radical trapping agents and membrane-protecting agents. It is the whole plant that is used.

The black tulip (Tulipa nigra) is the plant that contains the most plant DNA. It is a cultivated tulip. Its origin in the mountainous regions of Northern Europe and Asia explains its great qualities of resistance and reproduction, and also a great resistance to UV radiation. It is a petal extract that is used.

These plant extracts can be characterized by their solids content and/or by their active agent content.

The extracts are advantageously aqueous-glycolic or aqueous-alcoholic extracts, for example obtained by maceration under cold conditions in a water/propylene glycol or water/ethanol mixture.

Typically, the extracts given in detail hereinafter can be used for the preparation of the combination according to the present invention.

Extract of Gooseberry

| | |
|---|---|
| Water/ethanol | 50/50 |
| Dry material | 1.8% |
| Polyphenols (on a dry material basis) | 0.13% |
| Flavonoids (on a dry material basis) | 0.05% |

Extract of Black Orchid

| | |
|---|---|
| Water/propylene glycol | 50/50 |
| Dry material | 0.20% |
| Flavonoids (on a dry material basis) | 0.05% |

Extract of Black Tulip Petals

| | |
|---|---|
| Water/propylene glycol | 50/50 |
| Dry material | 0.6% |
| DNA | (presence) |

The combination can also be in the form of oily extracts that can be obtained by maceration of the plants in capric/caprylic triglycerides, oleic sunflower or any polar/apolar mixture of the apricot kernel oil/liquid petroleum jelly type. Liposoluble substances such as flavones and certain polyphenols can thus be extracted.

The topical compositions according to the invention may be in any cosmetic formulation.

The topical compositions according to the invention may, for example, comprise excipients suitable for external topical administration, in particular dermatologically acceptable excipients. These excipients suitable for the formulation are well known to those skilled in the art and comprise, in particular, thickeners such as natural gums and synthetic polymers; emollients and surfactants such as cetearyl octanoate, isopropyl myristate, cetearyl isononanoate, dimethicone, cyclomethicone, polyglyceryl 3-diisostearate, hydrogenated polyisobutene, cetyl alcohol, cetyl palmitate, cetyl phosphate; emulsifiers; preserving agents such as phenoxyethanol, methyl parahydroxybenzoate(methylparaben), propyl parahydroxybenzoate(propylparaben) and Phenonip® which combines phenoxyethanol and methyl, ethyl, butyl and isobutyl parahydroxybenzoates; colorants; fragrances; etc. Other ingredients may be used in the compositions: moisturizers such as propylene glycol, glycerol, butylene glycol and also anti-oxidizing vitamins such as vitamin E, for example tocopheryl acetate or tocotrienol, vitamin C, natural polyphenols. Skin-conditioning agents such as nylon can also be added to the composition.

The topical compositions according to the invention may be in the form of a solution, a hydrophilic lotion, an ointment, a cream, a serum or a gel.

The compositions may also be, for example, in the form of oil-in-water, water-in-oil or multiple emulsions or of foaming products or in a liposomal form.

All the compositions as described above can also be applied to the skin by means of wipes.

The topical compositions according to the invention may also include one or more of a diversity of optional ingredients, such as coloring agents, opacifiers, and the like.

In the combination according to the first aim of the invention, each of the gooseberry, black orchid and black tulip plant extracts is advantageously present at a content of between 5% and 90%, these contents being expressed relative to the total weight of the combination. Preferably, these contents are approximately 25% to 50%, and more preferentially, a combination comprising the three extracts in substantially equal amounts is used.

The topical compositions according to the invention typically include between 0.1% and 20% (w/w) by weight of the combination according to the present invention relative to the total weight of the topical composition, and preferably from 2% to 8%.

According to an advantageous embodiment, the extracts constituting the combination of the invention are completed with active ingredients or auxiliary ingredients chosen for their complementary properties, in order to reinforce the anti-ageing effects of the composition. Thus, it is particularly advantageous to combine them with appropriate amounts of an anti-hyaluronidase such as Echinacin B in order to generate a maximum amount of bound water, of Imperata cylindrica, for example MOIST 24® from the company Sederma, in order to promote moisturization of the skin by modification of the osmotic pressure, of amaranth seed proteins (Amarantus caudatus) or of pea globulins in order to obtain a skin-tightening effect, of Calophyllum oil in order to reinforce the antiwrinkle effect, of Echium oil for its anti-inflammatory effect, or else a palmitoylpentapeptide-3 such as Matrixyl® or derivatives such as palmitoyl GHK (having the chain Glycyl-Histidyl-Lysine) and palmitoyl GQPR (Glycyl-Glutamyl-Prolyl-Arginine) or palmitoyl VGVAPG (Valyl-Glycyl-Valyl-Alanyl-Prolyl-Glycine) combined with a ceramide-2 and also, in general, any combination with a ceramide.

The topical compositions according to the invention have an effect that improves skin damage due to ageing. The first results can be obtained after 2 to 3 weeks of daily treatment by applying the compositions once or twice a day to the areas of the skin to be treated.

Said compositions of the invention can be chosen for day and/or night use on the face, the body and the hands.

The compositions according to the invention are particularly suitable for the treatment of the areas around the eyes and the lips, which are very delicate and highly susceptible to the appearance of wrinkles and a loss of firmness of the skin. The compositions according to the invention are very well tolerated on this sensitive area, and make it possible to obtain a visible reduction in the number of wrinkles and marks around the eyes, and also a firming of the particularly sensitive skin around the eyes and the mouth.

The examples which follow illustrate the present invention without limiting the scope thereof.

Examples of formulations are reported in examples 1 to 4.

Example 5 relates to a toxicological study and to tests for activity on human fibroblasts, which made it possible to evaluate the anti-inflammatory effect (interleukins) and the anti-ageing effect (assay of MPP3, proteoglycans and collagens).

In examples 1 to 4 which follow, the “plant complex” corresponds to a combination based on gooseberry, black orchid and black tulip plant extracts according to the present invention in which the three extracts are those detailed above (extracted by maceration under cold conditions of a water/propylene glycol or water/ethanol mixture) and they are included in equal parts. Unless otherwise indicated, the compositions are given in parts by weight.

### EXAMPLE 1

### Soothing and Moisturizing Tonic Lotion

| | |
|---|---|
| Rose water | 5.0 |
| Cornflower water | 10.0 |
| Witch hazel water | 10.0 |
| 1,3-butylene glycol | 5.0 |
| Glycerol | 5.0 |
| Sodium lactate | 2.0 |
| Complex of plants according to the invention | 3.0 |
| Phenonip | 0.5 |
| Lettuce water | 5.0 |
| Demineralized water QS | 100.0 |

### EXAMPLE 2

### Moisturizing Radiance Day Cream

| | |
|---|---|
| Demineralized water QS | 100.0 |
| Glycerol | 5.0 |
| 1,3-butylene glycol | 4.0 |
| Complex of plants according to the invention | 5.0 |
| Yeast extract | 2.0 |
| Extract of Imperata cylindrica | 3.0 |
| Amaranth proteins | 1.0 |
| Hazelnut oil | 5.0 |
| Raspberry seed oil | 0.5 |
| Aminated cherry pit oil | 1.0 |
| Perhydrosqualene | 5.0 |
| Polysorbate 60 | 3.5 |
| Sorbitan stearate | 2.5 |
| Cetostearyl alcohol | 4.0 |
| Dimethicone | 1.0 |
| Tocopherols | 0.5 |

### EXAMPLE 3

### Regenerating Antiwrinkle Cream

| | |
|---|---|
| Demineralized water QS | 100.0 |
| Phenonip | 0.7 |
| Glycerol | 4.0 |
| 1,3-butylene glycol | 3.0 |
| 70% sorbitol | 2.0 |
| Complex of plants according to the invention | 10.0 |
| Matrixyl (palmitoyl pentapeptide-3)* | 3.0 |
| Amaranth proteins | 2.0 |
| Shear butter | 3.0 |
| Echium | 2.0 |
| Musk rose oil | 3.0 |
| Calophyllum oil | 2.0 |
| PEG 20 methyl glucose stearate | 3.5 |
| Methyl glucose stearate | 3.0 |
| Lecithin | 1.0 |
| Microcapsules of vitamin A and E | 1.0 |
| Magnesium ascorbyl sulfate | 0.3 |
| Propyl paraben | 0.1 |

*sold by Sederma

### EXAMPLE 4

### Regenerating Antiwrinkle Serum

Mixture of Lyophilized Plants:

| | |
|---|---|
| Gooseberry polyphenols | 0.05 |
| Extract of orchid | 0.10 |
| Black tulip DNA | 0.20 |
| Glycerine | 0.65 |
| TOTAL | 1.00 |

In a lyophilized flask

Solvent

| | |
|---|---|
| Glycerol | 0.5 |
| Butylene glycol | 0.5 |
| Biosaccharide gum-1 | 1.0 |
| Phenonip | 0.07 |
| pea globulins | 2.0 |
| Extract of Imperata cylindrica | 0.3 |
| Amaranth proteins | 1.0 |
| Demineralized water QS | 10.0 |

The lyophilized component is dissolved by the solvent and provides a treatment for 7 days.

### EXAMPLE 5

In addition to the toxicology of the product, studies on human fibroblasts in culture were carried out.

5-1. Experimental Protocol

1.1. Human Fibroblasts in Culture

The fibroblast cultures are established from skin from human foreskins taken during circumcisions, and are amplified in RPMI 1640 culture medium supplemented with fetal calf serum, L-glutamin and gentamycin. The fibroblasts were seeded into 25 cm² dishes in a proportion of 10⁶ cells per ml. They were then incubated for 24 hours.

1.2. Toxicological Aspect

The aim of this first step was to seek the cytotoxicity of the product with respect to human fibroblasts in culture. Cell growth is realized on three noncytotoxic concentrations.

The cytotoxicity was realized by determining protein neosynthesis.

The human fibroblasts obtained by primary culture were recovered after trypsinization and centrifugation. The cell pellet was resuspended in 10 ml of RPMI 1640 medium supplemented with fetal calf serum (10%), L-glutamin (8 mM) and gentamycin (80 μg/ml). After homogenization, the cells were distributed into multiwell plates (6-well) in a proportion of 1×10⁵ cells/ml. The plates were incubated for 24 hours. The medium was then removed and fresh medium was added, either alone, or supplemented with various concentrations of the “plant complex” product, 0.1%, 0.2%, 0.5%, 1% and 5%. The contact times of the product with the cells were 24 hours.

To study the ability of the cells, treated beforehand with the product, to incorporate radioactive precursors into proteins, the pulse technique was used. Radioactive precursors were added to the cultures after the incubation periods with the product at various concentrations. The cells were then incubated for a further two hours at 37° C. in the presence of ³H-leucin (1 μCi/ml of culture medium, specific activity: Ci/mmol).

After having removed the medium by suction, the cells were washed twice with serum-free medium in order to remove the nonincorporated radioactivity, and were then detached from the support by scraping the culture surface. The cells were washed once again and then centrifuged at 600 g for 5 minutes. The cell pellet was taken up in 500 μl of medium. To determine the radioactivity incorporated into the macromolecules, 500 μl of 20% (weight/volume) NaOH were added and the mixture was subjected to hydrolysis for 30 minutes at 37° C. The hydrolyzates were then precipitated with 1 ml of a 40% trichloracetic acid (TCA) solution. The preparations were left at 4° C. for one hour. The samples were filtered through a glass fiber filter (Whatman GF/A) mounted on a Millipore apparatus. The filters were washed three times with 10 ml of 5% TCA and twice with 95% ethanol and then introduced into counting flasks and dried for 30 minutes at 80° C.

The radioactivity was measured in a liquid scintillation counter in the presence of 5 ml of liquid scintillant (Ready organic, Beckman) containing 0.9% of acetic acid.

1.3. Assay of Inflammation Mediators (IL1-a, IL-6, TNF-α)

At the end of the incubation period (24 hours), the culture media were sampled and the assaying of IL1-a and of IL-6 was carried out in accordance with the protocols described in the assay kits:
interleukin 1-A (Il1-a) detection kit/R&D Systems
interleukin 6 (Il1-6) detection kit/R&D Systems

1.4. Evaluation of the Effect of Product on the Extracellular Matrix Constituents of Human Fibroblasts

1.4.1. Study of the Effect of the Product on Proteoglycans Synthesized by Human Fibroblasts

For this study, a large number of fibroblasts were used so as to have a sufficient amount of proteoglycans and of glycosaminoglycans. The fibroblasts were distributed into multiwell (6-well) dishes in a proportion of 2×10⁵/ml in 3 ml of culture medium. They were then maintained for 24 hours in CO₂ incubators.

In order to study the ability of the cells, treated beforehand with the substance, to incorporate the radioactive precursor, the pulse technique was used. The radioactive precursor ([3H]-glucosamine) was added to the cultures 18 hours before the recovery of the cells. The contact time of the cells with the product was 24 hours at 37° C.

After having removed the medium by suction, the cells were washed twice with serum-free medium in order to remove the nonincorporated radioactivity, and were then detached from the support by scraping the culture surface. The cells were washed once again with medium and then recentrifuged at 600 g for 5 minutes. The following were carried out on this final pellet:
assaying the proteoglycans by FPLC (Fast Protein Liquid Chromatography)
neosynthesis of total glycosaminoglycans (GAGS)
assaying of total proteins

Extraction

Perimembrane Proteoglycans

A first fraction of the pellet was resuspended in 1 M NaCl containing deoxyribonuclease (50 U/ml) and protease inhibitors. The homogenate was then incubated at 4° C. for 2 hours. Centrifugation for 30 minutes at 12 000 g made it possible to obtain the first homogenate containing the perimembrane proteoglycans. The pellet was used to extract the proteoglycans of the other two compartments (C1).

Transmembrane Proteoglycans

The pellet C1 obtained from the first extraction step was resuspended in 0.1% sodium azide containing sodium desoxycholate (DOC: 4%), sonicated at 75 mv for 20 seconds and then incubated for 2 hours at 4° C. Centrifugation for 30 minutes at 12 000 g made it possible to obtain the second supernatant containing the transmembrane proteoglycans. The pellet was used to extract the proteoglycans from the matrix compartment (C2).

Matrix Proteoglycans

The pellet C2 was washed three times with 0.1% sodium azide and was then resuspended, with agitation, in a 50 mM sodium acetate buffer containing 4 M guanidine HCl and 0.1% triton X-100 and also protease inhibitors.

Centrifugation for 30 minutes at 12 000 g made it possible to obtain the third supernatant containing the matrix proteoglycans.

Purification by FPLC

Prior to the purification by FPLC, each of the 3 supernatants was precipitated overnight at 4° C. from one volume of absolute ethanol, and then centrifuged at 12 000 g for 30 minutes. The pellets obtained were resuspended in a 50 mM tris-HCl buffer, pH 7.4.

The same analytical protocol was carried out for 3 solutes.

Anion exchange chromatography was used. Each pellet was resuspended in 250 ml of the 50 mM tris-HCl buffer, pH 7.4. Sepharose CL-6B (DEAE) gel (Pharmacia) was poured into a K 10/40 column (Pharmacia). The anion exchange gel has great resolution and a good yield. 100 μl of each sample were injected; the elution was monitored by detection with a spectrofluometer at a wavelength of 280 nm. The peak containing the proteoglycans was eluted at 1 M NaCl.

Assay

The assaying of the radioactivity is carried out at the HPLC outlet using a Packard counter (Flow-one).

1.4.2. Study of the Effect of the Product on Glycosaminoglycans Synthesized by Human Fibroblasts

A second fraction of the pellet was treated with 1 mg/ml pronase for 24 hours at 60° C. The reaction was stopped by cooling the tubes. The proteins were precipitated with 12% TCA at 4° C. overnight. The precipitates were then centrifuged at 12 000 g for 30 minutes. The supernatants containing the glycosaminoglycans were recovered, dialyzed against ultrapure water (MilliQ plus), and then lyophilized. The lyophilizates were then resuspended in a tris-HCl buffer pH 7.4, containing protease inhibitors. A 50 μl aliquot was removed for counting the radioactivity incorporated into the total glycosaminoglycans.

1.4.3. Study of the Effect of the Product on the Neosynthesis of Collagen Synthesized by Human Fibroblasts

For this study, the fibroblasts were cultured in the same way as in paragraph 1.4.1.

After the incubation period, the fibroblasts were recovered by centrifugation. The pellets were digested with collagens (1 mg/cell pellet) in 0.5 ml/l acetic acid for 24 hours at 4° C.

After centrifugation at 10 000 g, the collagens were precipitated with 1 M sodium chloride (NaCl), and the precipitate was resuspended and then dialyzed.

The primary amino acids were derivatized with o-phthaldehyde (OPA) acid, thus eliminating their interference. Hydroxyproline and proline were then derivatized with NBD-Cl by coupling the amino groups to NBD-CL. The NBD-Hyp was separated and identified by reverse-phase HPLC. For setting up the separation of the amino acid derivatives, coupling of a standard containing hydroxyproline to NBD-Cl is first carried out.
The hydroxyproline is assayed by measuring the fluorescence after reverse-phase HPLC separation:
   automatic injector
   ultrasep C18 column (30 cm×0.18 cm)
   6 μm porosity
   fluorescence detector.
The mobile phase consists of a mixture of acetonitrile/sodium phosphate buffer 0.1 mol/l, pH 7.2 (9:91 v/v), the flow rate is regulated at 1 ml/min, the elution is carried out in the static mode and the cycle is 10 minutes. The mobile phase is pre-filtered and then degassed before use.
The solutions are prepared in the following way: NBD-Cl: 25 mmol dissolved in methanol, OPA=150 mmol/l dissolved in methanol, Phosphate buffer=0.4 mmol/l, pH adjusted to 7.2.

The standard curve is prepared from a solution of hydroxyproline at 50 mg/l. Successive dilutions make it possible to obtain solutions ranging from 0.5 to 40 mg/l.

The derivatization and the establishment of the standard curve are carried out using 10 μl of a standard solution at various concentrations, mixed with 10 μl of the buffer. After the addition of 5 μl of OPA and agitation, the tubes are kept at ambient temperature for 5 min and then 10 μl of the NBD-Cl solution are added. The derivative is produced at 60° C., in a waterbath, for 3 minutes, in the dark. The tubes are then removed and the orange coloration makes it possible to verify the derivatization. They are, subsequently, placed in ice in order to ensure cooling. 50 μl of this mixture are then injected into the column in order to obtain the standard curve which must be linear.

The samples are treated in the same way.

1.4.4 Study of the Effect of the Product on MMP-3 (Metalloproteinase 3) Suppression

At the end of the incubation period (24 hours), the culture media were removed, and the MMP-3 assay was carried out in accordance with the protocols described in the assay kits.

Metalloproteinase 3 (MMP-3) detection kit: Interchim

2-Results

2.1. Evaluation of Cytotoxicity

The cytotoxicity was studied by incorporation of ³H-leucin into cell proteins after 24 hours of contact.

The results are given in the table below:

| | [³H]-leucin | |
|---|---|---|
| | incorporation (cpm) | % incorporation |
| Control | 5410 ± 450 | — |
| Plant complex (0.1%) | 5400 ± 570 | — |
| Plant complex (0.2%) | 5430 ± 120 | — |
| Plant complex (0.5%) | 5425 ± 230 | — |
| Plant complex (1%) | 5390 ± 120 | — |
| Plant complex (5%) | 4890 ± 220 | −9 (ns) |

2.2 Evaluation of Cell Growth
Cell Growth after 48 Hours of Contact

The results are given in the table below:

| | [³H]-leucin | |
|---|---|---|
| | incorporation (cpm) | % incorporation |
| Control | 13985 ± 950 | — |
| Plant complex | 13432 ± 120 | ns |
| (0.2%) | | |
| Plant complex | 13241 ± 230 | ns |
| (0.5%) | | |
| Plant complex | 13370 ± 120 | ns |
| (1%) | | |

ns: not significant

Cell Growth after 72 Hours of Contact

The results are given in the table below:

| | [³H]-leucin | |
|---|---|---|
| | incorporation | |
| Batches | (cpm) | % incorporation |
| Control | 17855 ± 1644 | — |
| Plant complex | 17855 ± 1343 | ns |
| (0.2%) | | |
| Plant complex | 17230 ± 1972 | ns |
| (0.5%) | | |
| Plant complex | 17534 ± 1612 | ns |
| (1%) | | |

ns: not significant

Comments

The results obtained show that the “plant complex” product at the concentrations of 0.2%, 0.5% and 1% has no effect on ³H-leucin incorporation into the proteins of human fibroblasts in culture after 48 and 72 hours of contact with the cells. The results reveal that the product is devoid of any cytotoxic activity at the concentrations used.

2.3 Evaluation of the Anti-Inflammatory Activity Interleukin 1-a Assay

The results are given in the table below:

| Batches | IL1-a (pg/ml) | % |
|---|---|---|
| Negative control | 59.8 ± 4.5 | — |
| UVB control | 118.2 ± 16.8 | +98 |
| (100 mJ/cm²) | | |
| Plant complex | 85.4 ± 11.8* | −27 |
| (0.2%) +UVB (100 mj/cm²) | | |
| Plant complex | 76.5 ± 15.5* | −35 |
| (0.5%) +UVB (100 mJ/cm²) | | |
| Plant complex | 72.8 ± 9.9* | −38 |
| (1%) +UVB (100 mJ/cm²) | | |

*significantly different with respect to the UVB-irradiated positive control (100 mJ/cm²) (p < 0.01)

Interleukin 6 Assay

The results are given in the table below:

| Batches | IL6 (pg/ml) | % |
|---|---|---|
| Negative control | 98.6 ± 6.5 | — |
| UVB Control | 165.2 ± 11.4 | +68 |
| (100 mJ/cm²) | | |
| Plant complex | 107.5 ± 9.5* | −35 |
| (0.2%) +UVB (100 mj/cm²) | | |
| Plant complex | 102.8 ± 6.6* | −37 |
| (0.5%) +UVB (100 mJ/cm²) | | |
| Plant complex | 105.3 ± 12.4* | −36 |
| (1%) +UVB (100 mJ/cm²) | | |

*significantly different with respect to the UVB-irradiated positive control (100 mJ/cm²) (p < 0.01)

Comments

The results obtained show that the UVB-irradiation of the fibroblasts (100 mJ/cm²) significantly increases the release of pro-inflammatory cytokines (IL-1a and IL6) into the culture medium respectively by +68%. The treatment of the cells with the “plant complex” product at the concentrations 0.2, 0.5 and 1% prior to the UVB-irradiation leads to a significant decrease in pro-inflammatory cytokines.

2.4. Evaluation of the Proteoglycan Content

Assaying of proteoglycans by incorporation of the ³⁵SO₄ radioactive precursor followed by estimation by FPLC.

Perimembrane Proteoglycans

The results are reported in the table below:

| | [³⁵SO₄] | |
|---|---|---|
| Batches | incorporation(cpm) | % incorporation |
| Control | 132 ± 33 | — |
| Plant complex | 221 ± 35* | +68 |
| (0.2%) | | |
| Plant complex | 258 ± 24* | +96 |
| (0.5%) | | |
| Plant complex | 261 ± 54* | +98 |
| (1%) | | |

*significantly different with respect to the control p < 0.01 (Wilcoxon Rank Sum Test)

Transmembrane Proteoglycans

The results are reported in the table below:

| | [³⁵SO₄] | |
|---|---|---|
| Batches | incorporation(cpm) | % incorporation |
| Control | 164 ± 22 | — |
| Plant complex | 203 ± 25* | +24 |
| (0.2%) | | |
| Plant complex | 242 ± 18* | +48 |
| (0.5%) | | |
| Plant complex | 249 ± 23* | +52 |
| (1%) | | |

*significantly different with respect to the control p < 0.01 (Wilcoxon Rank Sum Test)

Matrix Proteoglycans

The results are reported in the table below:

| | [³⁵SO₄] | |
|---|---|---|
| Batches | incorporation(cpm) | % incorporation |
| Control | 210 ± 27 | — |
| Plant complex | 268 ± 14* | +28 |
| (0.2%) | | |
| Plant complex | 298 ± 34* | +42 |
| (0.5%) | | |
| Plant complex | 285 ± 33* | +36 |
| (1%) | | |

*significantly different with respect to the control p < 0.01 (Wilcoxon Rank Sum Test)

Comments

The results obtained show that the “plant complex” product at the concentrations 0.2%, 0.5% and 1% significantly increases the perimembrane, transmembrane and matrix proteoglycan content.

2.5 Evaluation of the Collagen Content

The separation and the identification of hydroxyproline were carried out by reverse-phase HPLC. The fluorescence peaks, after integration, made it possible to calculate the concentration of hydroxyproline in the culture medium.

Perimembrane Proteoglycans

The results are reported in the table below:

| | [³⁵SO₄] | |
|---|---|---|
| Batches | incorporation(cpm) | % incorporation |
| Control | 1.85 ± 0.4 | — |
| Control + Vit C | 2.98 ± 0.48* | +61 |
| Plant complex | 2.24 ± 0.49* | +21 |
| (0.2%) | | |
| Plant complex | 2.54 ± 0.26* | +38 |
| (0.5%) | | |
| Plant complex | 2.63 ± 0.15* | +43 |
| (1%) | | |

*significantly different with respect to the control p < 0.01 (Wilcoxon Rank Sum Test)

Comments

The results obtained show that the “plant complex” product at the concentrations 0.2%, 0.5% and 1% significantly increases the collagen content of the human fibroblasts in culture (+21%, +38% and +43%), compared with vitamin C used as a positive reference (+61%).

2.6 Evaluation of the MMP3 Content

The MMP3 assay was carried out using ELISA kits on the culture medium.

The results are reported in the table below:

| | Concentration | |
|---|---|---|
| MMP3 | (MMP3)(pg/ml) | % |
| Control | 105.8 ± 10.6 | — |
| Plant complex | 84.4 ± 9.9* | −20 |
| (0.2%) | | |
| Plant complex | 75.5 ± 6.7* | −328 |
| (0.5%) | | |
| Plant complex | 62.2 ± 8.4* | −41 |
| (1%) | | |

*significantly different with respect to the control p < 0.01 (Wilcoxon Rank Sum Test)

Comments

The results obtained show that the “plant complex” product at the concentrations 0.2%, 0.5% and 1% decreases the expression of metalloproteinase 3.

1. A combination of plant extracts comprising gooseberry extract, black orchid extract and black tulip extract.

2. The combination as claimed in claim 1, wherein each of the gooseberry, black orchid and black tulip plant extracts is present at a content of between 5% and 90% by weight relative to the total weight of the combination.

3. The combination as claimed in claim 2, wherein the contents of each of the plant extracts are approximately 25% to 50% by weight relative to the total weight of the combination.

4. The combination as claimed in claim 3, wherein the three plant extracts are present in substantially equal amounts.

5. A topical composition for the treatment or prevention of damage to the skin associated with skin ageing, comprising a combination as claimed in claim 1.

6. The topical composition as claimed in claim 5, further comprising a component selected from the group consisting of an anti-hyaluronidase, Calophyllum oil, Echium oil, a palmitoyl pentapeptide-3 or derivatives thereof, a ceramide, and combinations thereof.

7. The use of a combination as claimed in claim 1 for the preparation of a topical composition for combating damage to the skin associated with skin ageing.

8. A cosmetic process for combating damage to the skin associated with skin ageing, comprising applying, to affected areas of the skin, a composition containing an effective amount of the topical composition as claimed in claim 5.

9. A cosmetic process for improving the external appearance, comprising applying a topical composition as claimed in claim 5.

10. A cosmetic process for combating damage to the skin associated with skin ageing comprising applying, to affected areas of the skin, a composition containing an effective amount of the topical composition as claimed in claim 6.

11. A cosmetic process for improving the external appearance, comprising applying a topical composition as claimed in claim 6.

12. The topical composition as claimed in claim 6, wherein the anti-hyaluronidase is selected from the group consisting of Echinacin B, Imperata cylindrica, amaranth seed proteins (Amarantus caudatus), pea globulins, and mixtures thereof.

13. The topical composition as claimed in claim 6, wherein the palmitoyl pentapeptide-3 derivatives are selected from the group consisting of palmitoyl GHK, palmitoyl GQPR, and palmitoyl VGVAPG combined with a ceramide-2.
